(19) United States
(12) Patent Application Publication
(10) Pub. No.: US 2004/0076651 A1
(43) Pub. Date: Apr. 22, 2004

(54) Cosmetic or dermatological agent in the form of a creamy permanent mousse or a stable foamed cream

(76) Inventors: Werner Brocks, Burghaun (DE); Heiko Eberhardt, Oberursel (DE); Axel Kalbfleisch, Darmstadt (DE); Karl-Heinz Kischka, Darmstadt (DE); Thomas Krause, Darmstadt (DE); Ernst Dieter Racky, Eppstein (DE)

Correspondence Address: Striker Striker & Stenby, 103 East Neck Road, Huntington, NY, 11743, US

(21) Appl. No.: 10/432,408

(22) PCT Filed: Oct. 18, 2001
(86) PCT No.: PCT/EP01/12062

(30) Foreign Application Priority Data
Nov. 24, 2000 (DE) 10058384.9

(51) Int. Cl. A61K 7/00

(52) U.S. Cl. 424/401

(57) ABSTRACT

Described is a cosmetic or dermatological agent in the form of a creamy permanent foam or in the form of a stable foamy cream which as a finished foam product is available in an appropriate container and can be dispensed therefrom. Preferred is a degree of foam-up of at least 10% and a density of the total mass of at the most 0.9 g/mL. The incorporated gas or the bubble structure produced remains stably contained in the mass even after storage of at least one week at room temperature (20° C.). The agent can be produced by preparing a liquid, foamable composition which at room temperature (20° C.) has a yield value, and subsequently or at the same time beating or foaming the composition with a gas.

The object of the invention is a cosmetic or dermatological agent, particularly a hair-care or skin-care product, in the form of a creamy permanent foam or of a stable foamy cream.

Cosmetic products must meet special requirements as regards the sensory properties conveyed to the user. Skin-care and hair-care products such as skin creams or hair-treatment agents are often in the form of creamy emulsions or dispersions and consist of an aqueous, hydrophilic phase and a hydrophobic phase containing oil or wax substances. The drawback of these current cosmetic care products in the form of emulsions or dispersions lies in their sensory and particularly tactile characteristics. These products often have a heavy, greasy and smeary feel. Many creams, because of the constituents they possess in order to be effective, have an unflattering, beige-colored or yellowish appearance. Additionally, in the case of hair-treatment agents and shampoos, improved spreadability on the hair and better emulsification when the agent is worked into moist hair are desirable. Hair-styling agents are often in the form gel-like products containing thickeners imparting an unpleasant, sticky feel.

Cosmetic hair-treatment products in the form of temporary, unstable foams generated just before use are known. They are either aerosol products dispensed from a pressurized container with the aid of a gas propellant and thus converted into an unstable foam, or they are what are known as pump foams which when dispensed from their container by means of a mechanical pump connected to a foam head produce an unstable foam. The drawback of products forming temporary foams is that the foam must be prepared before each use, which is very inconvenient for the user. Moreover, the required containers are expensive and prone to problems because of the danger of plugging of the product-dispensing system. Moreover, formulation freedom is restricted, meaning that not all desired skin-care and hair-care effects can be realized with temporary foam products.

Our task was to provide readily applicable cosmetic or dermatological skin-care and hair-care agents with improved sensory properties (for example, tactile, optical or acoustic properties) which would not unacceptably impair the primary skin-care or hair-care characteristics nor require expensive, problem-prone containers.

This objective is reached by means of a cosmetic or dermatological agent in the form of a creamy permanent foam or in the form of a stable foamy cream which is foamed in stable manner with a suitable gas, the degree of foam-up being at least 10%.

The object of the invention is a cosmetic or dermatological agent in the form of a creamy permanent foam or in the form of a stable foamy cream, said agent being available as a finished foam product in a suitable container from which it can be dispensed and being characterized in that the degree of foam-up after storage of at least one week at room temperature (20° C.) is still at least 10%.

The compositions of the agents of the invention are foamed in stable manner with air or an inert gas up to a degree of foam-up which typically is at least 10% and up to 500%, preferably between 20 and 200% and particularly between 30 an 100%. For purposes of the present invention, the degree of foam-up represents the volume ratio and is calculated from the density values of the composition before and after foaming, as follows:

[(D₀/D)−1]×100%

where D₀ denotes the density before foaming and D the density after foaming. Suitable gases for foaming, besides air, are in particular inert gases, for example nitrogen, carbon dioxide, nitrogen oxides, noble gases or mixtures of said gases. Of particular advantage for the production of foamed products containing oxidation-sensitive substances is the use of inert, oxygen-free gases such as nitrogen or carbon dioxide.

The concepts “stable foamy” and “permanent foam” refer to product compositions characterized in that in them is homogeneously distributed a gaseous substance in the form of small gas bubbles which remain in this homogeneous distribution over a period of at least one week, preferably at least one month and particularly at least 6 months if stored at room temperature (20° C.), namely the degree of foam-up is still at least 10% and preferably at least 20%. The foamed product compositions contain gas bubbles that preferably are from 0.0001 to 10 mm and particularly from 0.01 to 1 mm in size. The mean diameter of the gas bubbles is preferably from 0.1 to 0.8 mm and especially from 0.2 to 0.4 mm.

By introducing a gas into the basic composition, the density of the agent of the invention is adjusted to be preferably less than or equal to 0.9 g/mL, particularly 0.2 to 0.8 g/mL and especially 0.4 to 0.7 g/mL.

Suitable for preparing the agent of the invention are liquid, viscous, foamable compositions which at least at room temperature (20° C.) have a yield value. These compositions can consist of dispersions made up of a hydrophilic and a hydrophobic phase or they are a single-phase composition containing a thickener or a hydrocolloid.

The agent can be produced by (A) preparing a liquid, foamable composition which at room temperature (20° C.) has a yield value and (B) subsequently or at the same time beating or foaming the composition with air and/or an inert gas to a degree of foam-up of at least 10%.

In particular, agents according to the invention can be produced by

(A) either preparing a liquid, dispersed composition containing a hydrophilic phase, a hydrophobic phase and at least one surfactant, the hydrophobic phase containing at least one substance that is solid at room temperature (20° C.), or by preparing a liquid, foamable aqueous composition containing a combination of at least one thickener and at least one surfactant, and subsequently or at the same time

(B) beating or foaming the composition with air and/or an inert gas,

the air and/or inert gas introduced or the bubble structure formed remaining stable in the mass even after storage of at least one month at room temperature (20° C.).

Another object of the invention is a product prepared in this manner, namely a cosmetic or dermatological agent in the form of a creamy permanent foam or in the form of a stable foamy cream which contains a hydrophilic phase, a hydrophobic phase, at least one surfactant and at least one gas stably introduced into the composition or at least one thickener, at least one surfactant and at least one gas stably introduced into the composition.

Preferred agents contain

(a) water

(b) at least one substance which at room temperature (20° C.) is solid, waxy or greasy

(c) at least one surfactant

(d) at least one foam stabilizer and

(e) at least one skin-care or hair-care substance.

Hydrophilic Phase

The expression “aqueous phase” or “hydrophilic phase” includes water as well as mixtures of water and water-soluble, cosmetically compatible organic solvents, for example the lower monohydric or polyhydric alcohols, particularly polyhydric C₂ to C₄ alcohols, for example ethylene glycol, diethylene glycol, butylene glycol or glycerol. The lower monohydric alcohols such as ethanol and isopropanol, because of their foam-inhibiting action, should not be used at all or should be used only in very small amounts that do not impair foaming.

Hydrophobic Phase

The expression “hydrophobic phase” refers to a phase containing a hydrophobic, water-insoluble wax, fat or oil. At room temperature, such substances can be liquid or solid and contain at least one hydrophobic substance which is solid at room temperature (20° C.), because the stability of the foamed product is thus enhanced.

The hydrophobic substances which are solid at room temperature (20° C.) are added either in the molten form or they are added as solids to the hydrophilic phase that has been heated to above the solid's melting point. These substances can be waxes or wax-like materials, for example natural renewable waxes (insect, animal and plant waxes), fossil waxes (petroleum waxes, lignite waxes, peat waxes or ozokerites), synthetic waxes (Fischer-Tropsch, polyethylene or amide waxes) high-melting paraffins, esters, fats, long-chain carboxylic acids or long-chain C₁₀ to C₂₂ alcohols, each having a melting point or solidification point above room temperature (20° C.).

In addition to the hydrophobic solid, the hydrophobic phase can contain hydrophobic substances that are still liquid at room temperature. These include oils or oil-like substances, for example naturally occurring, renewable oils (plant and animal fatty oils), synthetic oils, silicone oils, mineral oils, essential oils, water-insoluble, branched or linear aliphatic hydrocarbons, linear or branched alcohols, particularly liquid fatty alcohols, and long-chain ethers and esters, the said substances preferably having at least eight carbon atoms. Suitable hydrocarbons are, for example, liquid paraffins, squalane or squalene. Also suitable are the esters of trihydric and polyhydric alcohols, particularly the plant triglycerides such as, for example, olive oil, almond oil, walnut oil and sunflower oil, and the synthetic triglycerides, for example C₈-C₁₀-trifatty acid esters of glycerol, or jojoba oil.

Also suitable as the hydrophobic substance are the monoesters or diesters having the formulas R¹—COOR², R¹—COO—R³—OOCR¹ and R²OOC—R³—COOR² wherein R¹ denotes a C₈ to C₂₂-alkyl group, R² denotes a C₃ to C₂₂-alkyl group and R³ denotes a C₂ to C₁₆-alkylene group. Also suitable are naturally occurring monoester or wax ester mixtures, such as, for example, the branched primary alcohols present in jojoba oil or sperm oil, known as Guerbet alcohols.

Other suitable hydrophobic substances are materials which are usually employed as opacifying agents for cosmetic products, particularly those having the formula R¹—COO—(CHR⁴CHR⁵O)ₙ—COR⁶ wherein R¹ denotes a C₈ to C₂₂-alkyl group, R⁴ and R⁵ denote hydrogen or methyl and R⁶ denotes hydrogen or R¹ and n is a numeral from 1 to 12, preferably 1, 2, 3 or 4. Preferred are glycol difatty acid esters and polyethylene glycol difatty acid esters that are solid at room temperature.

The quantity of the hydrophobic phase in the unfoamed dispersion depends on the requirements placed on the end product to be produced. For example, for hair treatments it can amount to from 5 to 30 wt % and for skin creams or hair creams even up to about 50 wt %. Correspondingly, the hydrophilic phase then amounts to 50 to 95 wt %. At any rate, it is advisable to select the kind and amount of the dispersed substances so that, as will be described in greater detail hereinbelow, a sufficiently high viscosity and a yield value are attained for both the unfoamed and the foamed compositions.

A special embodiment of the invention consists of hair-cleaning and body-cleaning agents in the form of a creamy permanent foam or in the form of a stable foamy cream containing water, at least one detersive surfactant, at least one hydrophobic, wax-like substance that is solid at room temperature (20° C.) and at least one gas stably introduced into the composition, the gas introduced or the bubble structure formed remaining stable in the mass for at least one month during storage at room temperature (20° C.).

Surfactants

The homogenization of the hydrophilic and hydrophobic phases can be accomplished without an emulsifier. Preferably, however, at least one surfactant is used as dispersing aid. Foaming then produces a fine-cell foam. The surfactant can be used in an amount from 0.1 to 30 wt % and preferably from 0.2 to 5 wt %, based on the unfoamed composition. Higher amounts of surfactant, for example from 5 to 25 wt %, are used when the agent of the invention is employed as skin-cleansing or hair-cleaning agent (shampoo).

Suitable surfactants are nonionic, anionic, cationic, amphoteric or zwitterionic surfactants. The surfactants indicated, for example, in the “International Cosmetic Ingredient Dictionary and Handbook”, 7th edition, vol. 2, in the section on “Surfactants”, especially in the subsection on “Surfactants—Emulsifying Agents”, are suitable.

Nonionic surfactants are, for example, ethoxylated fatty alcohols, ethoxylated nonylphenols, alkyl polyglycosides, fatty monoglycerides and diglycerides, ethoxylated and hydrogenated or non-hydrogenated castor oil, fatty alkanolamides and ethoxylated fatty esters. Cationic surfactants are, for example, the long-chain quaternary ammonium compounds known under the CTFA designation of “quaternium”, for example the alkyltrimethylammonium salts or dialkyldimethylammonium salts with C₈ to C₂₂-alkyl groups. Suitable cationic surfactants can be represented by general formula (I)

N⁽⁺⁾R¹R²R³R⁴X⁽⁻⁾ (I)

wherein R¹ to R⁴ independently of each other denote aliphatic groups, aromatic groups, alkoxy groups, polyoxyalkylene groups, alkylamido groups, hydroxyalkyl groups, aryl groups or alkaryl groups, each with 1 to 22 carbon atoms, at least one of the R¹ to R⁴ groups having at least 8 carbon atoms, and X⁽⁻⁾ denotes a cosmetically compatible anion, for example a halogen, acetate, phosphate, nitrate or alkylsulfate and preferably a chloride. Besides the carbon atoms and hydrogen atoms, the aliphatic groups can also contain cross-links or other groups, for example other amino groups. Examples of suitable cationic surfactants are the chlorides or bromides of alkyldimethylbenzylammonium salts, alkyltrimethylammonium salts, for example cetyltrimethylammonium chloride or bromide, tetradecyltrimethylammonium chloride or bromide, alkyldimethylhydroxyethylammonium chlorides or bromides, dialkyldimethylammonium chlorides or bromides, alkylpyridinium salts, for example laurylpyridinium or cetylpyridinium chloride, alkylamidoethyltrimethylammonium ether sulfate and compounds of a cationic nature such as amine oxides, for example alkylmethylamine oxides or alkylaminoethyldimethylamine oxides. Particularly preferred is cetyltrimethylammonium chloride. Suitable cationic surfactants are, in particular, also the so-called esterquats, for example the C₈ to C₁₈ alkyl esters of betaine, for example palmitylbetaine chloride.

Suitable anionic surfactants, particularly for use in the hair-cleaning and body-cleaning agents of the invention, are selected from among the alkali metal or alkaline earth metal salts of the C₁₀ to C₁₈ alkylsulfates, the C₁₀ to C₁₈ alkylsulfonates, the C₁₀ to C₁₈ alkylbenzenesulfonates, the C₁₀ to C₁₈ xylenesulfonates and the C₁₀ to C₁₈ alkyl ether sulfates ethoxylated with 1 to 10 ethylene oxide units, the ethoxylated sulfosuccinate half-esters of general formula

R—(OCH₂CH₂)ₘ—O₂C—CH₂—CH—(SO₃M)—COOM

wherein R denotes a C₁₀ to C₁₈ alkyl group, M denotes an alkali metal cation or alkaline earth metal cation, and m is a numeral from 1 to 10, and the alkyl ether carboxylates of general formula

R—(OCH₂CH₂)ₙ—OCH₂—COOM

wherein R denotes a C₁₀ to C₁₈ alkyl group, M denotes an alkali metal cation or alkaline earth metal cation and n is a numeral from 1 to 20, the alkali metal salts and alkaline earth metal salts of C₁₀ to C₁₈ alkyl ether sulfates ethoxylated with 1 to 10 ethylene oxide units being particularly preferred.

Suitable amphoteric surfactants, particularly for use in the hair-cleaning and body-cleaning agents of the invention, are selected from among derivatives of aliphatic quaternary ammonium, phosphonium and sulfonium compounds of the general formula

R¹—Y⁽⁺⁾(R²)ₓ—CH₂—R—Z⁽⁻⁾

wherein R¹ denotes a linear or branched alkyl, alkenyl or hydroxyalkyl group with 8 to 18 carbon atoms, with 0 to 10 ethylene oxide units and with 0 to 1 glycerol unit; Y denotes an N-, P- or S-containing group; R² denotes an alkyl or monohydroxyalkyl group with 1 to 3 carbon atoms; x equals 1 when Y is an S-atom and x equals 2 when Y is an N-atom or P-atom; R³ denotes an alkyl or hydroxyalkyl group with 1 to 4 carbon atoms, and Z denotes a carboxylate, sulfate, phosphonate or phosphate group. Other suitable amphoteric surfactants are those derived from betaine, for example the C₈ to C₁₈-alkylbetaines such as cocodimethylcarboxymethylbetaine, lauryldimethylcarboxymethylbetaine, lauryidimethyl-alpha-carboxyethylbetaine, cetyldimethylcarboxymethylbetaine, oleyldimethyl-gamma-carboxypropylbetaine, or lauryl-bis-(2-hydroxypropyl)-alpha-carboxyethylbetaine; C₈-C₁₈-alkylsulfobetaines such as cocodimethylsulfopropylbetaine, stearyidimethylsulfopropylbetaine, lauryldimethylsulfoethylbetaine, lauryl-bis-(2-hydroxyethyl)sulfopropylbetaine; the carboxyl derivatives of imidazole, the C₈-C₁₈-alkyldimethylammonium acetates, the C₈-C₁₈-alkyldimethylcarbonylmethylammonium salts as well as the C₈-C₁₈ fatty acid alkylamidobetaines, for example, coco fatty acids amidopropylbetaine (INCI designation: cocoamidopropylbetaine) and the N-coco fatty acid amidoethyl-N-[2-(carboxymethoxy)ethyl]glycerol (INCI designation: cocoamphocarboxyglycinate),

Foam Stabilizers, Thickeners, Hydrocolloids

Foamed products with improved stability can be obtained when the compositions contain at least one foam stabilizer, said stabilizer being capable of keeping the gas stably dispersed in the foamed composition. The foam stabilizers are preferably used in an amount from 0.05 to 30 wt % and particularly from 0.2 to 10 wt %.

Suitable foam stabilizers are hydrocolloids and thickeners, particularly those conferring a yield value to the composition. Suitable stabilizers are

synthetic polymers such as, for example, polyvinylpyrrolidone or the cross-linked polyacrylates (Carbomers, Carbopols),

polymers of natural origin, particularly polysaccharides and derivatives thereof, for example sclerotium gum, starch, gelatin, cellulose and derivatives thereof such as carboxymethylcellulose, hydroxypropylcellulose, methylcellulose, hydroxypropylmethylcellulose or hydroxyethylcellulose, microcrystalline cellulose and extracts of algae such as agar, carrageenan or the alginates, as well as carouba gum, guar gum and the derivatives thereof, for example, alkylated or hydroxyalkylated guar, karaya gum, xanthan gum, gum arabic, and pectins,

inorganic thickeners such as the hectorites, bentonites, aluminum silicates and magnesium silicates,

or a mixture of said substances.

Particularly preferred thickeners are the cross-linked polyacrylic acids and their salts, the polysaccharides, polysaccharide derivatives and agar.

Skin-Care and Hair-Care Additives

The agent of the invention advantageously contains additionally hair-care or skin-care ingredients in an amount from, for example, 0.01 to 10 wt % and preferably from 0.1 to 5 wt %, based on the unfoamed composition. Suitable hair-care additives are, in particular, plant and herbal extracts, protein and silk hydrolyzates, sunscreen agents, antioxidants, free-radical scavengers, antidandruff agents, luster-imparting agents, vitamins, panthenol, softeners, combability improvers etc. Suitable combability improvers are cationic or cation-active hair-care agents, for example, cationic polymers, cationic surfactants, cationic silicone compounds, cationically derivatized proteins or protein hydrolyzates and betaines, with at least one cationic or cation-active group each.

Suitable skin-care or dermatologically active substances are, for example, softening, moisturizing or moisture-retaining substances, inflammation-inhibiting substances, sunscreen agents, vitamins, proteins, insect repellents, bactericides, virucides, antimicrobial, proteolytic or keratolytic substances, medicinals etc.

Preparation

The unfoamed dispersion can be prepared by known methods. A review of modern methods for preparing semi-solid and liquid emulsions is provided in an article published in SÖFW Journal 124, May 1998, pages 308 to 313 and in an article published in SÖFW Journal 118, May 1992, pages 287 to 296. As a rule, the preparation is carried out by heating the hydrophobic fatty phase to about 75° C. and combining it, by intensive mixing in a mixer or homogenizer, with the hydrophilic aqueous phase, also heated to 75° C. The finished dispersion is then cooled. Gas is preferably introduced at a time when the mixture has not yet cooled completely, for example at 30-40° C.

Subsequently or at the same time, in a beater (for example Euromix or the Top Mix dynamic foam generator supplied by Hansa Industrie Mixer, or an Ultra Turax laboratory mixer) the dispersion is beaten with a gas, preferably air, CO₂ or nitrogen. Particularly advantageous is the use of nitrogen. The foam rise preferably amounts to 20 to 200% and makes it possible to affect the structure and consistency in a desirable manner. Preferred are mixers consisting of a conveying system and a rotor/stator mixing head to which a gas can be supplied during the mixing process by means of an additional connection. Particularly preferred is a Becomix Duohomogenizer which has an additional connection through which gas can be supplied directly to the rotor, the gas then coming in contact with the phase contained in the mixer only in the ring gears of the homogenizer. The gas can be supplied either by reducing the pressure in the mixing tank (vacuum) and/or by increasing the pressure in the gas supply line. By changing the transported quantity, rotational speed, temperature, pressure and amount of gas supplied, it is possible to adjust the foam density, consistency, viscosity and foam bubble size as desired.

The stably foamed products are placed into suitable containers, for example jars or tubes. The containers are preferably made of a transparent material, for example glass or a transparent plastic material, so as to show off the product's advantageous optical properties. The permanent foams are storage-stable over a long time and present a creamy to cream-like or velvety feel, depending on the foam bubble size.

Consistency, Viscosity

The end product is a viscous composition for which the desired final viscosity is usually already attained by homogenization. The desired final viscosity, however, can also be achieved by addition of an electrolyte such as sodium chloride or sodium sulfate or of some other substance with thickening action such as cellulose or a cellulose derivative.

For particularly good stability of the foamed products, both the unfoamed and the foamed composition should present a yield value and a sufficiently high viscosity. The viscosity of the unfoamed composition is preferably in the range from 1000 to 30,000 mPa s and especially from 3000 to 20,000 mPa s, and the viscosity of the foamed composition is preferably between 1000 and 100,000 mPa s and particularly between 1500 and 80,000 mPa s, as measured with a Haake VT 550 viscometer, measuring system SVDIN, at 25° C. and a shear gradient of 50 s⁻¹. The yield value is preferably at least 10 Pa and particularly at least 50 Pa, as measured with the Bohlin Rheometer CS, measuring system CP 4/40, at 25° C. and with a shear stress rising linearly from 0.1 to 600 Pa (200 s).

The products foamed according to the invention are characterized by the fact that they contain very small, uniformly distributed gas bubbles. The agents exhibit unusually advantageous sensory properties:

They exert a very pleasant tactile effect and have a creamy or velvety but not sticky feel, they have a low density, they are light-weight and they are easily spread on keratinic surfaces such as the skin and hair;

They have a pleasant, cosmetic, white color of unusual purity; in particular, they make it possible to improve the appearance of a cream which in the unfoamed state has a beige or yellowish color, because even minor amounts of very finely distributed air bubbles make the cream appear whiter;

Depending on the foam stabilizer used and the foam bubble size chosen, acoustic effects are also possible, for example a crackling noise when the foams are squeezed out of the tubes or during the spreading on the skin or while working the product into the hair.

The agents also have the advantage that they can be readily applied to inclined surfaces without running off or dripping.

Preferred embodiments are

a permanently foamed cosmetic skin cream containing at least one skin-care substance,

a permanently foamed dermatological or medicinal ointment containing at least one active ingredient with action on the skin or a pharmaceutical or dermatological active ingredient which the body can absorb through the skin;

a permanently foamed suncream containing at least one organic or inorganic UV filter;

a permanently foamed hair-styling cream containing at least one hair-firming substance;

a permanently foamed hair-treatment cream containing at least one hair-care substance;

a permanently foamed permanent wave or hair-straightening cream containing at least one active ingredient with permanent waving or hair-straightening action.

### EXAMPLES

### Example 1

### Foamed Cationic Hair Treatment

A dispersion with the following constituents was prepared.

| | | |
|---|---|---|
| 12.0 | g | of cetearyl alcohol |
| 2.7 | g | of petrolatum |
| 2.4 | g | of mineral oil |
| 2.2 | g | of cetyltrimethylammonium chloride |
| 0.6 | g | of lanolin alcohol |
| 0.5 | g | of citric acid |
| 0.3 | g | of lanolin |
| 0.0012 | g | of tocopherol |
| to 100 | g | water |

The dispersion was foamed with nitrogen to a degree of foam-up of 23 to 27% with the Top Mix dynamic foam generator (Hansa Industrie Mixer).

### Example 2

### Foamed Cationic Hair Treatment

A dispersion with the following constituents was prepared.

| | | |
|---|---|---|
| 6.0 | g | of cetearyl alcohol |
| 1.35 | g | of petrolatum |
| 1.2 | g | of mineral oil |
| 1.1 | g | of cetyltrimethylammonium chloride |
| 1.0 | g | of sclerotium gum |
| 0.5 | g | of citric acid |
| 0.5 | g | of perfume |
| 0.3 | g | of lanolin alcohol |
| 0.15 | g | of lanolin |
| 0.0006 | g | of tocopherol |
| to 100 | g | water |

The dispersion was foamed with nitrogen to a degree of foam-up of 23 to 27% with the Top Mix dynamic foam generator (Hansa Industrie Mixer).

### Example 3

### Foamed Cationic Hair Treatment

A dispersion with the following constituents was prepared.

| | | |
|---|---|---|
| 6.0 | g | of cetearyl alcohol |
| 1.35 | g | of petrolatum |
| 1.2 | g | of mineral oil |
| 1.1 | g | of cetyltrimethylammonium chloride |
| 1.0 | g | of sclerotium gum |
| 0.5 | g | of polyvinylpyrrolidone |
| 0.5 | g | of citric acid |
| 0.5 | g | of perfume |
| 0.3 | g | of lanolin alcohol |
| 0.15 | g | of lanolin |
| 0.0006 | g | of tocopherol |
| to 100 | g | water |

The dispersion was foamed with nitrogen to a degree of foam-up of 23 to 27% with the Top Mix dynamic foam generator (Hansa Industrie Mixer).

### Example 4

### Foamed Skin Cream

A dispersion with the following constituents was prepared.

| | | |
|---|---|---|
| 5.0 | g | of mineral oil |
| 9.0 | g | of glyceryl stearate |
| 2.0 | g | of beeswax |
| 1.5 | g | of Cutina ® BW-V (INCI: glyceryl hydroxystearate and |
| | | cetyl esters and microcrystalline wax and |
| | | trihydroxystearin and hydroxystearic acid and |
| | | tetradecyloctadecanoic acid and |
| | | tetradecyleicosanoic acid and hexadecyleicosanoic acid) |
| 1.5 | g | of stearic acid |
| 0.3 | g | of preservative (methylparaben, propylparaben) |
| 0.2 | g | of perfume |
| to 100 | g | water |

The dispersion was foamed with nitrogen with a Top Mix dynamic foam generator (Hansa Industrie Mixer).

### Example 5

### Foamed Anionic Cream Base for Hair-Treatment Products

A dispersion with the following constituents was prepared.

| | | |
|---|---|---|
| 2.0 | g | of PEG-3 distearate |
| 2.0 | g | of ethylene glycol distearate |
| 5.5 | g | of glyceryl stearate |
| 17.5 | g | of Lanette ® W (90% of cetearyl alcohol, |
| | | 10% of lauryl sulfate) |
| 2.0 | g | of lanolin alcohol |
| 10.0 | g | of lauryl ether sulfate (28% in water) |
| 0.44 | g | of Na cocoylisethionate |
| 1.7 | g | of sodium sulfate |
| to 100 | g | water |

The dispersion was foamed with nitrogen with a Top Mix dynamic foam generator (Hansa Industrie Mixer). The density before foaming was 1.02 kg/L. The foaming was carried to a density of 0.50 kg/L. This corresponds to a degree of foam-up of 104%. The composition could be foamed further to a density of 0.35 kg/L. This corresponds to a degree of foam-up of 191%.

### Example 6

### Foamed Cream Base for Hair-Treatment Products

| | | |
|---|---|---|
| 5.0 | g | of liquid paraffin |
| 9.0 | g | of glyceryl stearate |
| 2.0 | g | of beeswax, bleached |
| 1.5 | g | of Cutina ^{®} BW-V |
| 1.5 | g | of stearic acid |
| to 100 | g | water |

The raw materials were weighed and heated to 80° C. The water was also heated to 80° C. The water phase was emulsified into the fatty phase, The foaming was done in a Turrax laboratory mixer the gasket of which was removed so that air could be drawn into the mass during mixing. The mass was foamed with air at 8000 rpm.

The density of the finished foam was 0.577 g/cm³. The viscosity of the unfoamed composition was 4617 mPa s, and the viscosity of the foamed composition was 1583 mPa s, measured with a Haake VT 550 viscometer, measuring system SVDIN, at 25° C. and a shear gradient of 50 s⁻¹, The yield value of the foamed composition was 524 Pa measured with the Bohlin Rheometer CS, measuring system FL/DG, at 25° C. and a shear stress rising linearly from 0.25 to 1000 Pa (200 s).

### Example 7

### Shampoo

A dispersion with the following constituents was prepared.

| | | |
|---|---|---|
| 20 | g | of PEG-3 distearate |
| 13.7 | g | of sodium lauryl ether sulfate |
| 2.1 | g | of cocamidopropylbetaine |
| 0.24 | g | of sodium benzoate |
| 0.2 | g | of sodium citrate |
| 0.15 | g | of sodium formate |
| 0.15 | g | of benzoic acid |
| 0.12 | g | of Luviquat ® FC 370 (BASF, INCI; polyquaternium-16) |
| 0.1 | g | of panthenol |
| 0.02 | g | of formic acid |
| to 100 | g | water |

The mixture of constituents was heated to 75° C. During cooling, the mixture was treated 10 minutes at 8000 rpm with the Turrax (slide bearing, no gasket) and foamed with the stirrer until it solidified.

| | | |
|---|---|---|
| Density before foaming: | 0.96 | g/cm³ |
| Density 9 days after foaming: | 0.61 | g/cm³ |
| Degree of foam-up after 9 days: | 57% | |
| Yield value before foaming: | 92.6 | Pa |
| Yield value 9 days after foaming: | 104 | Pa |
| Viscosity before foaming: | 14.700 | mPa s |
| Viscosity 9 days after foaming: | 58.800 | mPa s |

The viscosity was measured with the Haake VT 550 viscometer, measuring system SVDN, at 25° C. and a shear gradient of 50 s⁻¹. The yield value was measured with a Bohlin Rheometer CS, measuring system CP 4/40, at 25° C. and a shear stress rising linearly from 0.1 to 600 Pa (200 s).

### Example 8

### Permanent Foams Based on Thickeners and Surfactants

| | A | B | C | D | E |
|---|---|---|---|---|---|
| Carbopol | 0.5 g | 0.5 g | 0.5 g | — | — |
| Agar | — | — | — | 4.0 g | — |
| Lattice ® NT-050⁽¹⁾ | — | — | — | — | 4.0 g |
| Oramix ® NS 10⁽²⁾ | 0.5 g | — | — | 0.5 g | 0.5 g |
| Laureth-4 | — | 0.5 g | — | — | — |
| Tween 40⁽³⁾ | — | — | 0.5 g | — | — |
| Water | to 100 g | to 100 g | to 100 g | to 100 g | to 100 g |

⁽¹⁾Microcrystalline cellulose, FMC Corp.
⁽²⁾Decyl glucoside, Seppic
⁽³⁾Polysorbate-40, ICI Surfactants

The thickeners were dissolved in water. To this end, Carbopol was 100% neutralized with aminomethylpropanol (AMP). Agar was predissolved with heating and then cooled to about 55° C. After addition of the surfactant, the mixture was foamed and then cooled in a refrigerator. The compositions were foamed with air by vigorous stirring with a commercial laboratory mixer. The foamed products showed good to very good optical foam quality and good foam stability. The best results were obtained with Oramix NS 10 as the surfactant. The use of agar led to a product with unusually advantageous tactile properties. It produced a special, velvety feel when spread on the skin.

1. Cosmetic or dermatological agent in the form of a creamy permanent foam or in the form of a stable foamy cream, said agent being available as a finished foam product in an appropriate container from which it can be removed, characterized in that the degree of foam-up after storage of at least one week at room temperature (20° C.) is still at least 10%.

2. Agent according to claim 1, characterized in that the foam bubble size is from 0.0001 to 10 mm.

3. Agent according to one of the preceding claims, characterized in that the degree of foam-up is from 10 to 500%.

4. Agent according to one of the preceding claims, characterized in that the density of the mass is adjusted to a value less than or equal to 0.9 g/mL by introducing a gas.

5. Agent according to one of the preceding claims, characterized in that it is foamed with a gas selected from among air, nitrogen, carbon dioxide, nitrogen oxides, noble gases or a mixture of said gases.

6. Agent according to one of the preceding claims, characterized in that it is produced by
(A) preparing a liquid, foamable composition which at room temperature (20° C.) has a yield value, and subsequently or at the same time
(B) beating or foaming the composition with air and/or an inert gas,
the degree of foam-up after storage of at least one week at room temperature (20° C.) being at least 10%.

7. Agent according to one of the preceding claims, characterized in that it is produced by
(A) either preparing a liquid, foamable composition containing a hydrophilic phase, a hydrophobic phase and at least one surfactant, the hydrophobic phase containing at least one substance which is solid at room temperature (20° C.) or by preparing a liquid, foamable aqueous composition containing a combination of at least one thickener and at least one surfactant, and subsequently or at the same time,
(B) beating or foaming the composition with air and/or an inert gas,
the degree of foam-up after storage of at least one week at room temperature (20° C.) being at least 10%.

8. Agent according to one of the preceding claims containing a dispersed composition which contains a hydrophilic phase, a hydrophobic phase, at least one surfactant, at least one hair-care or skin-care substance and at least one gas stably incorporated into the mass, wherein the hydrophobic phase contains at least one substance that is solid at room temperature (20° C.).

9. Agent according to claim 8, characterized in that the hydrophobic phase contains at least one wax, fat or oil component selected from among natural waxes, fossil waxes, synthetic waxes, paraffins, fatty acids, fatty alcohols, esters of fatty acids, esters of fatty alcohols, plant oils; animal-oils, synthetic oils, mineral oils, essential oils, water-insoluble, branched or linear aliphatic-hydrocarbons, wherein the said substances preferably contain at least 12 carbon atoms, or silicone oils, monoesters or diesters having the formulas R¹—COOR², R¹—COO—R³—OOCR¹ and R²OOC—R³—COOR² wherein R¹ denotes a C₈ to C₂₂-alkyl group, R² denotes a C₃ to C₂₂-alkyl group and R³ denotes a C₂ to C₁₆-alkylene group.

10. Agent according to one of claims 8 or 9, characterized in that the dispersed composition contains at least one foam stabilizer.

11. Agent according to one of claims 1 to 7 containing a liquid, foamable aqueous composition which contains a combination of at least one thickener, at least one surfactant, at least one hair-care or skin-care substance and at least one gas stably incorporated into the mass.

12. Agent according to one of the preceding claims in the form of a body-cleaning or hair-cleaning agent containing water, at least one detersive surfactant, at least one hydrophobic, wax-like substance that is solid at room temperature and at least one gas stably incorporated into the mass, wherein the degree of foam-up even after storage of at least one week at room temperature (20° C.) is at least 10%.

13. Agent according to one of the preceding claims, characterized in that it contains a foam stabilizer selected from among hydrocolloids and thickeners, particularly polyvinylpyrrolidone, cross-linked or uncross-linked polyacrylic acids or the salts thereof, sclerotium gum, starch, gelatin, cellulose and derivatives thereof, agar, carrageenan, alginates, carouba gum, guar gum and the derivatives thereof, karaya gum, xanthan gum, gum arabic, pectins, hectorites, bentonites, aluminum silicates and magnesium silicates,

14. Agent according to claim 13, characterized in that the foam stabilizer is selected from among cross-linked and uncross-linked polyacrylic acids or the salts thereof, polysaccharides, polysaccharide derivatives and agar.

15. Agent according to one of the preceding claims, characterized in that it contains at least one skin-care or hair-care additive selected from among plant extracts or herbal extracts, protein and silk hydrolyzates, sunscreen agents; antioxidants, free-radical scavengers, antidandruff agents, luster-imparting agents, vitamins, panthenol, softeners, combability improvers, skin softeners, moisturizing or moisture-retaining substances, inflammation-inhibiting substances, proteins, vitamins, insect repellents, bactericides, virucides, antimicrobial, proteolytic or keratolytic substances and medicinals.

16. Use of a finished foam product available in an appropriate container and which after storage of at least one week at room temperature (20° C.) still has a degree of foam-up of at least 10%, for hair-care, hair styling, hair and body cleaning or skin care.

17. Use of a finished foam product available in an appropriate container and which after storage of at least one week at room temperature (20° C.) still has a degree of foam-up of at least 10% for preparing a permanently foamed cosmetic skin cream containing at least one skin-care substance; of a permanently foamed dermatological or medicinal ointment containing at least one active ingredient with action on the skin or a pharmaceutical or dermatological active ingredient which the body can absorb through the skin; of a permanently foamed suncream containing at least one organic or inorganic UV filter; of a permanently foamed hair styling cream containing at least one hair-firming substance; of a permanently foamed hair-treatment cream containing at least one hair-care substance or of a permanently foamed permanent wave or hair-straightening cream containing at least one active ingredient with permanent waving or hair-straightening action.

18. Method for preparing an agent according to one of claims 1 to 15, characterized in that the foaming is accomplished by passing the liquid, unfoamed composition through a mixer fitted with a conveying system, a mixing head and an additional connection for feeding a gas, and at the same time supplying a gas to the mixer through the additional connection.
